(19) United States
(12) Patent Application Publication
(10) Pub. No.: US 2016/0144039 A1
(43) Pub. Date: May 26, 2016

(54) METHODS FOR ENHANCING STABILITY OF POLYORTHOESTERS AND THEIR FORMULATIONS

(71) Applicant: Heron Therapeutics, Inc., Redwood City, CA (US)

(72) Inventors: Devang T. Shah, Fremont, CA (US); John Barr, Half Moon Bay, CA (US)

(21) Appl. No.: 14/946,493

(22) Filed: Nov. 19, 2015

Related U.S. Application Data
(63) Continuation of application No. 14/133,300, filed on Dec. 18, 2013, now abandoned.
Application No. 14/133,300 is a continuation of application No. 12/635,681, filed on Dec. 10, 2009, now abandoned.
(60) Provisional application No. 61/121,894, filed on Dec. 11, 2008.

(51) Int. Cl. A61K 47/34; A61K 47/10; A61K 31/439

(52) U.S. Cl. CPC A61K 47/34; A61K 31/439; A61K 47/10

(57) ABSTRACT

Disclosed herein are methods of enhancing the stability of a sustained pharmaceutical composition comprising an active agent and a polymer and methods of preparing such pharmaceutical compositions with enhanced stability.

### CROSS-REFERENCE TO RELATED APPLICATIONS

This application is a continuation of U.S. patent application Ser. No. 14/133,300, filed Dec. 18, 2013, which is a continuation of U.S. patent application Ser. No. 12/635,681, filed Dec. 10, 2009, now abandoned, which claims the benefit of U.S. Provisional Patent Application No. 61/121,894, filed Dec. 11, 2008, all of which are incorporated herein by reference in their entirety.

### BACKGROUND OF THE INVENTION

The present application relates to novel methods for enhancing the stability of controlled release polymers and their pharmaceutical compositions. In one embodiment, the pharmaceutical composition comprises a polyorthoester and an active ingredient. In another embodiment, the present application discloses pharmaceutical compositions prepared according to the methods as described in the present application.

### DESCRIPTION OF THE ART

Interest in synthetic biodegradable polymers for the systemic delivery of therapeutic agents began in the early 1970's with the work of Yolles et al., Polymer News 1:9-15 (1970) using polylactic acid). Since that time, numerous other polymers have been prepared and investigated as bioerodible matrices for the controlled release of therapeutic agents.

U.S. Pat. Nos. 4,079,038, 4,093,709, 4,131,648, 4,138,344 and 4,180,646 disclose biodegradable or bioerodible polyorthoesters. These polymers are formed by a reaction between an ortho ester (or orthocarbonate) such as 2,2-diethoxytetrahydrofuran and a diol such as 1,4-cyclohexanedimethanol. The reaction requires elevated temperature and reduced pressure and a relatively long reaction time. Drugs or other active agents are retained in the polymer matrix. The agents are released as the polymer biodegrades due to hydrolysis of the labile linkages.

U.S. Pat. No. 4,304,767 discloses polymers prepared by reacting a polyol with a polyfunctional ketene acetal. These polymers represent a significant improvement over those of U.S. Pat. Nos. 4,079,038, 4,093,709, 4,131,648, 4,138,344 and 4,180,646, since synthesis proceeds readily at room temperature and atmospheric pressure, and the resulting polymers are disclosed to have superior properties.

Further polymers are disclosed in U.S. Pat. No. 4,957,998. These polymers contain acetal, carboxy-acetal and carboxy-ortho ester linkages, and are prepared by a two-step process beginning with the reaction between a polyfunctional ketene acetal and a compound containing a vinyl ether, followed by reaction with a polyol or polyacid. Still further polymers of a similar type are disclosed in U.S. Pat. No. 4,946,931. The polymers are formed by a reaction between a compound containing a multiplicity of carboxylate functions and a polyfunctional ketene acetal. The resulting polymers have very rapid erosion times.

Despite the ease with which the ortho ester linkage hydrolyses, polyorthoesters known in the art are extremely stable materials when placed in an aqueous buffer, or when residing in the body. This stability is attributable to the extreme hydrophobicity of the polyorthoesters which severely limits the amount of water that can penetrate the polymer. To achieve useful erosion rates, therefore, acidic excipients must be physically incorporated into the polymer. While this allows control over erosion rates, the physically incorporated acidic excipient can diffuse from the polymer matrix at varying rates, leaving a matrix that is completely depleted of excipient while the polymer still has a very long lifetime remaining.

U.S. Pat. No. 5,968,543 discloses polyorthoester polymers useful as orthopedic implants or vehicles for the sequestration and sustained delivery of drugs, cosmetic agents and other beneficial agents through incorporation of esters of short-chain α-hydroxy acids such as esters of glycolic acid, lactic acid or glycolic-co-lactic acid copolymer into the polyorthoester polymer chain and variation of the amount of these esters relative to the polymer as a whole.

In the presence of water, these esters, when incorporated into the polymer chain, are readily hydrolyzed at a body temperature of 37° C. and a physiological pH, in particular at a pH of 7.4, to produce the corresponding α-hydroxy acids. The α-hydroxy acids then act as an acidic excipient to control the hydrolysis rate of the polymer. When the polymer is used as a vehicle or matrix entrapping an active agent, the hydrolysis of the polymer causes release of the active agent.

U.S. Patent Publication No. 2007/0265329 discloses pharmaceutical compositions and methods for the sustained and controlled release of an effective amount of a selective 5-hydroxytryptamine 3 (5-HT₃) receptor antagonist, comprising a polyorthoester and a pharmaceutically acceptable liquid excipient. The (5-HT₃) receptor antagonists are useful for the prevention, reduction or alleviation of acute and delayed chemotherapy-induced nausea and vomiting (CINV) following a course of emetogenic chemotherapy.

The patents and references listed in this section and elsewhere throughout this application are incorporated herein by reference in their entirety.

### SUMMARY OF THE INVENTION

In one aspect, this present application provides a method of enhancing the stability of a sustained release pharmaceutical composition comprising an active agent and a polymer, wherein the method comprises heating the pharmaceutical composition at an elevated temperature for a sufficient period of time to provide a more stable pharmaceutical composition than that of the unheated pharmaceutical composition when stored at room temperature. In another aspect, the present application discloses a method of enhancing the stability of the polymer, wherein the method comprises heating the polymer at an elevated temperature for a sufficient period of time to provide a more stable polymer than that of the unheated polymer, when stored at room temperature.

In another aspect, the present application provides a method of enhancing the stability of a sustained release pharmaceutical composition comprising an active agent and a polyorthoester polymer, wherein the method comprises treating the pharmaceutical composition under a condition comprising one or more of the following: an elevated temperature, a sufficient period of time, an inert gas, and a reduced pressure. In another aspect, the present application provides a method of enhancing the stability of a sustained release pharmaceutical composition consisting essentially of a polyorthoester polymer without an active agent, wherein the method comprises treating the pharmaceutical composition under a condition comprising one or more of the following: an elevated temperature, a sufficient period of time, an inert gas and a reduced pressure.

In another aspect, this present application provides a method of preparing a sustained release pharmaceutical composition with enhanced stability wherein the method comprises treating the pharmaceutical composition under a condition comprising one or more of the following: an elevated temperature, a sufficient period of time, an inert gas, and a reduced pressure, and wherein the pharmaceutical composition comprises an active agent and a polymer.

These and other embodiments are further described in the text that follows.

### BRIEF DESCRIPTION OF THE FIGURES

FIG. 1 depicts the average molecular weight (Mw) of a pharmaceutical composition (APF530, Formulation F) stored in syringes at about 5° C. over time, wherein the pharmaceutical composition comprises granisetron and a polyorthoester Polymer A as defined below.

FIG. 2 depicts the average molecular weight (Mw) of the above pharmaceutical composition stored in syringes at 40° C. over time.

FIG. 3 shows the pharmacokinetic profile of the above composition (APF530) administered in the clinic to healthy humans.

FIG. 4 shows the pharmacokinetic profile of the above composition (APF530) administered in the clinic to chemotherapy patients.

FIG. 5 shows the profiles of the in vitro release of granisetron from the above composition (APF530) stored in syringes at 25° C. for up to 24 months.

FIG. 6 shows the average molecular weight of the above composition (APF530) stored in syringes at 5, 25, 30 and 40° C. for up to 24 months.

FIG. 7 shows profiles of the in vitro release of granisetron from a composition with enhanced stability (APF530R, Formulation FR) stored in syringes at 25° C. for up to 9 months.

FIG. 8 shows the average molecular weight of the above composition with enhanced stability (APF530R, Formulation FR) stored in syringes 5, 25, 30 and 40° C. for up to 9 months.

FIG. 9 shows the profiles of the in vitro release of buprenorphine from a pharmaceutical composition (APF580R) wherein the pharmaceutical composition comprises buprenorphine and a polyorthoester Polymer A as defined below stored in syringes at 25° C. for up to 15.5 months.

FIG. 10 shows the average molecular weight of the above composition (APF580R) stored in syringes at 5, 25, 30 and 40° C. for up to 15.5 months.

FIG. 11 shows the pharmacokinetic profile of several buprenorphine compositions (with and without enhanced stability) administered to male and female beagle dogs.

### DETAILED DESCRIPTION OF THE INVENTION

### Definitions

Unless defined otherwise in this specification, all technical and scientific terms are used herein according to their conventional definitions as they are commonly used and understood by those of ordinary skill in the art of synthetic chemistry and pharmacology.

“Stability” as used herein refers to the ability of a polymer composition or a pharmaceutical composition described herein to remain in a steady state wherein the properties of the pharmaceutical composition remain substantially unchanged under a specific condition, such as under a specific storage condition, during the period between the time it is prepared in a form ready for administration to a patient and the time when it is administered to a patient. The properties of a pharmaceutical composition of this invention may include, but is not limited to, the viscosity of the composition, the molecular weight of the polymer and/or rate of release of the active agent, etc. A pharmaceutical composition is substantially stable under the specified conditions, as provided herein, for example, when the composition does not degrade or undergo hydrolysis to any appreciable extend over an extended period of time under the specified storage conditions. A pharmaceutical composition is in a steady state (or is stable) when the properties of the pharmaceutical composition do not vary more than 10%, more than 5%, more than 3% or more than 2% over a period of four weeks, eight weeks, six months, twelve months or longer under the specified storage conditions. In one aspect, the compositions do not vary more than 3% over at least four weeks. Accordingly, in one aspect, there is provided a method of enhancing the stability of the composition of the present application so that the properties of the composition do not vary more than 10%, more than 5%, more than 3% or more than 2% over a period of four weeks, eight weeks, six months, twelve months or longer under the specified storage conditions.

A “polymer susceptible to hydrolysis” refers to a polymer that is capable of degradation, disassembly or digestion through reaction with water molecules. Such a polymer contains hydrolyzable groups, such as an ester group, in the polymer. Examples of polymers susceptible to hydrolysis may include, but is not limited to, polyorthoester, such as those described herein, and those described in U.S. Pat. Nos. 4,079,038, 4,093,709, 4,131,648, 4,138,344, 4,180,646, 4,304,767, 4,957,998, 4,946,931 and 5,968,543, and U.S. Patent Publication No. 2007/0265329, which are incorporated by reference in their entirety.

“Bioerodible” and “bioerodibility” refer to the degradation, disassembly or digestion of a polymer by action of a biological environment, including the action of living organisms and most notably at physiological pH and temperature. As an example, a principal mechanism for bioerosion of a polyorthoester is hydrolysis of linkages between and within the units of the polyorthoester.

“Comprising” is an inclusive term interpreted to mean containing, embracing, covering or including the elements listed following the term, but not excluding other unrecited elements.

“Sustained release”, “extended release” and similar terms are used to denote a mode of active agent delivery that occurs when the active agent is released from the delivery vehicle at an ascertainable and controllable rate over a period of time, rather than dispersed immediately upon application or injection. Extended or sustained release, which may also be controlled using the methods taught in the present invention, may extend for hours, days or months, and may vary as a function of numerous factors. For the pharmaceutical composition of the present application, the rate of release will depend on the type of the excipient selected and the concentration of the excipient in the composition. Another determinant of the rate of release is the rate of bioerosion of the polymer used in the composition. The rate of bioerosion is primarily controlled by hydrolysis of the polymer which in turn may be controlled by the composition of the polyorthoester and the number of hydrolyzable bonds in the polyorthoester. Other factors determining the rate of release of an active ingredient from the present pharmaceutical composition include particle size, solubility of the active agent, acidity of the medium (either internal or external to the matrix) and physical and chemical properties of the active agent in the matrix, as well as the biological environment, such as pH and temperature. As used herein, “controlled release” used in combination with “sustained release” of the pharmaceutical composition also means that the specific profile of the release of the active agent, such as granisetron or buprenorphine, in addition to the sustained or extended release period, may be controlled to provide optimum efficacy with the desired therapeutic effects.

“Delivery vehicle” denotes a composition which has the functions including transporting an active agent to a site of interest, controlling the rate of access to, or release of, the active agent by sequestration or other means, and facilitating the application of the agent to the region where its activity is needed. In the pharmaceutical compositions described herein, the delivery vehicle comprises a polymer susceptible to hydrolysis, such as a polyorthoester. The delivery vehicle of certain pharmaceutical composition further comprises an excipient compatible with the polymer.

“Polyorthoester-compatible” refers to, in one particular aspect of the properties of the polyorthoester, the properties of an excipient which, when mixed with the polyorthoester, forms a single phase and does not cause any physical or chemical changes to the polyorthoester.

“Semi-solid” denotes the mechano-physical state of a material that is flowable under moderate stress. More specifically, the semi-solid material should have a viscosity between about 10,000 and 3,000,000 cps, especially between about 30,000 and 500,000 cps. Preferably the composition or formulation is easily syringable or injectable, meaning that it can readily be dispensed from a conventional tube of the kind well known for topical or ophthalmic formulations, from a needleless syringe, or from a syringe with a 16 gauge or smaller needle, such as 16-25 gauge.

“Sequestration” is the confinement or retention of an active agent within the internal spaces of a polymer matrix. Sequestration of an active agent within the matrix may limit the toxic effect of the agent, prolong the time of action of the agent in a controlled manner, permit the release of the agent in a precisely defined location in an organism, or protect unstable agents against the action of the environment.

An “active agent” or “active ingredient” refers to any compound or mixture of compounds which produces a beneficial or useful result. Active agents are distinguishable from such components as vehicles, carriers, diluents, lubricants, binders and other formulating aids, and encapsulating or otherwise protective components. Examples of active agents are pharmaceutical, agricultural or cosmetic agents. Suitable pharmaceutical agents include locally or systemically acting pharmaceutically active agents which may be administered to a subject by topical or intralesional application (including, for example, applying to abraded skin, lacerations, puncture wounds, etc . . . , as well as into surgical incisions) or by injection, such as subcutaneous, intradermal, intramuscular, intraocular or intra-articular injection. Suitable pharmaceutical agents include polysaccharides, DNA and other polynucleotides, antisense oligonucleotides, antigens, antibodies, vaccines, vitamins, enzymes, proteins, naturally occurring or bioengineered substances, and the like, anti-infectives (including antibiotics, antivirals, fungicides, scabicides or pediculicides), antiseptics (e.g., benzalkonium chloride, benzethonium chloride, chlorhexidine gluconate, mafenide acetate, methylbenzethonium chloride, nitrofurazone, nitromersol and the like), steroids (e.g., estrogens, progestins, androgens, adrenocorticoids and the like), opioids (e.g. buprenorphine, butorphanol, dezocine, meptazinol, nalbuphine, oxymorphone and pentazocine), therapeutic polypeptides (e.g. insulin, erythropoietin, morphogenic proteins such as bone morphogenic protein, and the like), analgesics and anti-inflammatory agents (e.g., aspirin, ibuprofen, naproxen, ketorolac, COX-1 inhibitors, COX-2 inhibitors and the like), antipsychotic agents (for example, phenothiazines including chlorpromazine, triflupromazine, mesoridazine, piperacetazine and thioridazine; thioxanthenes including chlorprothixene and the like), antiangiogenic agents (e.g., combresiatin, contortrostatin, anti-VEGF and the like), anti-anxiety agents (for example, benzodiazepines including diazepam, alprazolam, clonazepam, oxazepam; and barbiturates), anti-depressants (including tricyclic antidepressants and monoamine oxidase inhibitors including imipramine, amitriptyline, doxepin, nortriptyline, amoxapine, tranylcypromine, phenelzine and the like), stimulants (for example, methylphenidate, doxapram, nikethamide and the like), narcotics (for example, buprenorphine, morphine, meperidine, codeine and the like), analgesic-antipyretics and anti-inflammatory agents (for example, aspirin, ibuprofen, naproxen and the like), local anesthetics (e.g., the amide- or anilide-type local anesthetics such as bupivacaine, dibucaine, mepivacaine, procaine, lidocaine, tetracaine and the like), fertility control agents, chemotherapeutic and anti-neoplastic agents (for example, mechlorethamine, cyclophosphamide, 5-fluorouracil, thioguanine, carmustine, lomustine, melphalan, chlorambucil, streptozocin, methotrexate, vincristine, bleomycin, vinblastine, vindesine, dactinomycin, daunorubicin, doxorubicin, tamoxifen and the like), cardiovascular and anti-hypertensive agents (for example, procainamide, amyl nitrite, nitroglycerin, propranolol, metoprolol, prazosin, phentolamine, trimethaphan, captopril, enalapril and the like), drugs for the therapy of pulmonary disorders, anti-epilepsy agents (for example, phenytoin, ethotoin and the like), anti-hidrotics, keratoplastic agents, pigmentation agents or emollients, antiemetic agents (such as ondansetron, granisetron, tropisetron, metoclopramide, domperidone, scopolamine and the like). The composition of the present application may also be applied to other locally acting active agents, such as astringents, antiperspirants, irritants, rubefacients, vesicants, sclerosing agents, caustics, escharotics, keratolytic agents, sunscreens and a variety of dermatologies including hypopigmenting and antipruritic agents. The term “active agents” further includes biocides such as fungicides, pesticides and herbicides, plant growth promoters or inhibitors, preservatives, disinfectants, air purifiers and nutrients. Pro-drugs and pharmaceutically acceptable salts of the active agents are included within the scope of the present application.

A “therapeutically effective amount” means the amount that, when administered to an animal for treating a disease, is sufficient to effect treatment for that disease.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

### Methods

Sustained release pharmaceutical compositions comprising certain polymers as a delivery vehicle to provide controlled and/or sustained release of an active pharmaceutical agent in a patient display instability when stored at ambient temperature. Such instability can be in the form of reduced molecular weight, reduced viscosity, and/or increased release of active agent. For example, the pharmaceutical composition in the Formulation in the Example below, showed reduction of molecular weight and viscosity and increase in in vitro release of active agent when stored at 40° C. over a two-four week period. However, when the same formulation is stored at 2-8° C. for at least one year, its molecular weight and viscosity remain substantially unchanged. Therefore, to avoid degradation of the pharmaceutical composition during storage and transportation, such pharmaceutical compositions must be kept under refrigeration conditions, resulting in increased storage and transportation costs. Further, if the stringent storage conditions are not met, the composition may be degraded so that it does not provide the same active agent release rate as desired, which may result in the administration of an improper or undesired amount of active agent to a patient.

In one embodiment, there is provided a method of enhancing the stability of a sustained release pharmaceutical composition comprising an active agent and a biocompatible polymer. In some embodiments, the polymer is a polymer that is susceptible to hydrolysis. In some embodiments, the method comprises reducing the water content in the pharmaceutical composition or reducing the water content in the polymer. In some embodiments, the water content is reduced by treating the pharmaceutical composition under a condition comprising one or more of the following: an elevated temperature, a sufficient period of time, an inert gas and/or a reduced pressure. In some embodiments, the condition comprises an elevated temperature and a sufficient period of time. In some embodiments, the condition comprises an elevated temperature, a sufficient period of time and an inert gas. In some embodiments, the condition comprises an elevated temperature, a sufficient period of time and a reduced pressure.

In some embodiments, the method comprises heating the pharmaceutical composition at an elevated temperature for a sufficient period of time to provide a more stable pharmaceutical composition than that of the unheated pharmaceutical composition when stored at room temperature. That is, the pharmaceutical composition obtained from the above heat treated method results in a composition that is more stable than the same pharmaceutical composition that has not been heat treated, when the two compositions are stored at room temperature for the same amount of time, under the same conditions.

In another aspect, this application provides a method of enhancing the stability of a sustained release pharmaceutical composition comprising an active agent and a polyorthoester polymer, wherein the method comprises treating the pharmaceutical composition under a condition comprising one or more of the following: an elevated temperature, a sufficient period of time, an inert gas and a reduced pressure. In some embodiments, the condition comprises an elevated temperature and a sufficient period of time. In some embodiments, the condition comprises an elevated temperature, a sufficient period of time and an inert gas. In some embodiments, the condition comprises an elevated temperature, a sufficient period of time and a reduced pressure. In another aspect, the present application provides a method of enhancing the stability of a pharmaceutical composition comprising a polyorthoester polymer, wherein the method comprises treating the pharmaceutical composition under a condition comprising one or more of the following: an elevated temperature, a sufficient period of time, an inert gas and a reduced pressure. In some embodiments, the condition comprises an elevated temperature and a sufficient period of time. In some embodiments, the condition comprises an elevated temperature, a sufficient period of time and an inert gas. In some embodiments, the condition comprises an elevated temperature, a sufficient period of time and a reduced pressure.

In yet another aspect, this application provides a method of preparing a sustained release pharmaceutical composition with enhanced stability wherein the method comprises treating the pharmaceutical composition under a condition comprising one or more of the following: an elevated temperature, a sufficient period of time, an inert gas, and a reduced pressure, and wherein the pharmaceutical composition comprises an active agent and a polyorthoester polymer. In some embodiments, the condition comprises an elevated temperature and a sufficient period of time. In some embodiments, the condition comprises an elevated temperature, a sufficient period of time and an inert gas. In some embodiments, the condition comprises an elevated temperature, a sufficient period of time and a reduced pressure.

In some embodiments of the above methods, the elevated temperature is at least about 40° C., at least about 50° C., at least about 70° C. or at least about 80° C. In some embodiments, the elevated temperature is from about 80° C. to about 120° C. In some embodiments, the elevated temperature is from about 85° C. to about 100° C. In some embodiments, the elevated temperature is from about 90° C. to about 95° C.

In some embodiments of the above methods, the period of time is at least 12 hours. In certain variations, the period of time is at least 12 hours at the above cited elevated temperatures. In one embodiment of the above, the period of time is at least 24 hours. In one embodiment of the above methods, the inert gas is argon, or the inert gas is nitrogen.

In some embodiments of the above methods, the reduced pressure is at or below about 50% of the atmospheric pressure. In some embodiments, the reduced pressure is at or below about 25% of the atmospheric pressure. In some embodiments, at or below about 10% of the atmospheric pressure. In some embodiments, at or below about 1% of the atmospheric pressure.

In some embodiments of the above methods, the molecular weight of the polymer is reduced. In some embodiments, the molecular weight is reduced by at least about 5%, at least about 10%, at least about 20%, at least about 30%, at least about 40%, or at least about 50% as compared with the molecular weight before the treatment.

In some embodiments of the above methods, the viscosity of the pharmaceutical composition is reduced. In some embodiments, the viscosity is reduced by at least about 5%, at least about 10%, at least about 20%, at least about 30%, at least about 40%, or at least about 50% as compared with the viscosity of the pharmaceutical composition that is not heat treated as disclosed above.

In some embodiments of the above methods, the release rate of active agent in the pharmaceutical composition is increased. In some embodiments, the release rate is increased by at least about 5%, at least about 10%, at least about 20%, at least about 30%, at least about 40%, or at least about 50% as compared with the release rate before the treatment.

In certain cases, the pharmaceutical composition described herein is loaded in a syringe, sealed and stored. Thus, in some embodiments of this application, the method comprises treating a pharmaceutical composition under a condition comprising one or more of the following: an elevated temperature, a sufficient period of time, an inert gas, and a reduced pressure, loading the treated pharmaceutical composition to a syringe, and sealing the syringe. In some embodiments, the method further comprises storing the syringe loaded with the treated pharmaceutical composition under ambient temperature for an extended period of time wherein the pharmaceutical composition remains stable for its intended use. Such extended period can be at least four weeks, at least eight weeks, at least three months, at least six months, at least twelve months, at least eighteen months, or at least two years.

### Polymers

It is contemplated that the method of the present application is useful in enhancing the stability of a pharmaceutical composition comprising a polymer. In one aspect of the present method, the pharmaceutical composition comprises the polymer without an active agent. The polymers that may be stabilized with the methods of the present application include a polymer that is capable of degradation, disassembly or digestion through reaction with water molecules, such as a polymer having hydrolyzable groups, such as an ester group, in its polymer chain structure. Examples of polymers susceptible to hydrolysis may include, but is not limited to, polyorthoesters, such as those described herein, and those described in U.S. Pat. Nos. 4,079,038, 4,093,709, 4,131,648, 4,138,344, 4,180,646, 4,304,767, 4,957,998, 4,946,931 and 5,968,543, and U.S. Patent Publication No. 2007/0265329, which are incorporated by reference in their entirety.

In some embodiments of the above methods, the polymer is a polyorthoester of formula I, formula II, formula III or formula IV

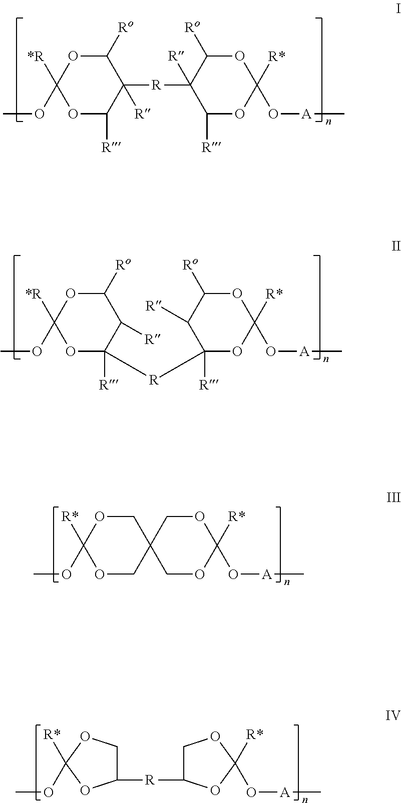

where:

R is a bond, —(CH₂)_{a—}, or —(CH₂)_{b}—O—(CH₂)_{c}—; where a is an integer of 1 to 10, and b and c are independently integers of 1 to 5;

R* is a C₁₋₄ alkyl;

R^{o}, R″ and “‘are each independently H or C₁₋₄ alkyl;

n is an integer of at least 5; and

A is R¹, R², R³, or R⁴, where

R¹ is:

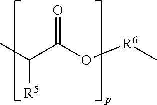

where:

p is an integer of 1 to 20;

R⁵ is hydrogen or C₁₋₄ alkyl; and

R⁶ is:

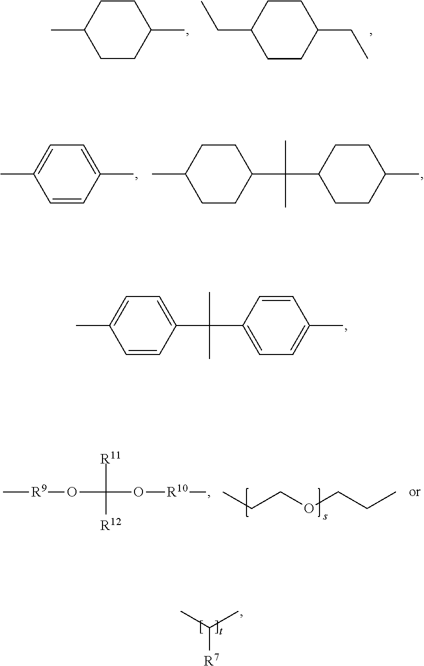

where:

s is an integer of 0 to 30;

t is an integer of 2 to 200; and

R⁷ is hydrogen or C₁₋₄ alkyl;

R² is:

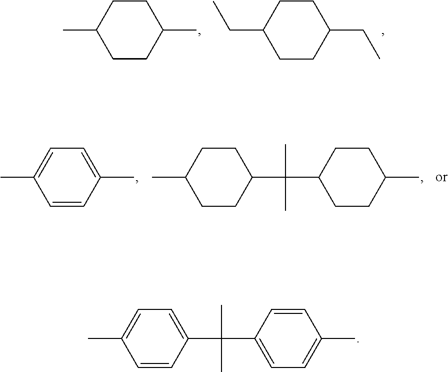

### R³ is:

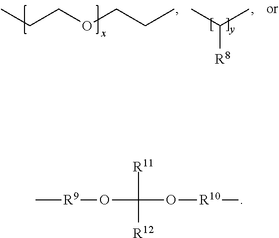

where:

x is an integer of 0 to 100;

y is an integer of 2 to 200;

R⁸ is hydrogen or C₁₋₄ alkyl;

R⁹ and R¹⁰ are independently C₁₋₁₂ alkylene;

R¹¹ is hydrogen or C₁₋₆ alkyl and R₁₂ is C₁₋₆ alkyl; or R¹¹ and R¹² together are C₃₋₁₀ alkylene; and

R⁴ is the residue of a diol containing at least one functional group independently selected from amide, imide, urea, and urethane groups;

in which at least 0.01 mol percent of the A units are of the formula R¹.

In some embodiments, A is R¹ R³, or R⁴, where

R¹ is:

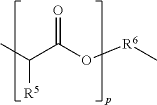

where:

p is an integer of 1 to 20;

R³ and R⁶ are each independently:

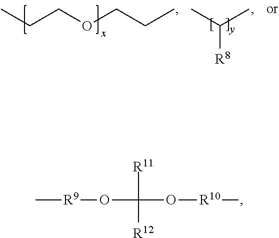

where:

x is an integer of 0 to 30;

y is an integer of 2 to 200;

R⁸ is hydrogen or C₁₋₄ alkyl;

R⁹ and R¹⁰ are independently C₁₋₁₂ alkylene;

R¹¹ is hydrogen or C₁₋₆alkyl and R¹² is C1-6 alkyl; or R¹¹ and R¹² together are C₃₋₁₀ alkylene;

R⁴ is a residual of a diol containing at least one functional group independently selected from amide, imide, urea and urethane groups; and R⁵ is hydrogen or C₁₋₄ alkyl; and in which at least 0.01 mol percent of the A units are of the formula R¹.

In another embodiment, the concentration of the polyorthoester ranges from 1% to 99% by weight. In yet another embodiment, the polyorthoester has a molecular weight between 3,000 and 10,000. In another embodiment, the fraction of the A units that are of the formula R¹ is between 5 and 15 mole percent.

In another embodiment of the above method, the polyorthoester is of formula I, where: none of the units have A equal to R²;

R³ is:

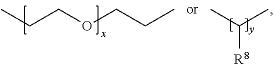

where:

x is an integer of 0 to 10;

y is an integer of 2 to 30; and

R⁶ is:

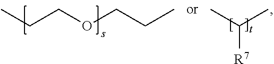

where:

s is an integer of 0 to 10;

t is an integer of 2 to 30; and

R⁵, R⁷, and R⁶ are independently hydrogen or methyl.

In another embodiment of the above method, R³ and R⁶ are both —(CH₂—CH₂—O)₂—CH₂—CH₂)—; R⁵ is methyl; and p is 1 or 2. In another embodiment, R³ and R⁶ are both —(CH₂—CH₂—O)_{9—} (CH₂—CH₂)—; R⁵ is methyl; and p is 1 or 2. In another variation, the polyorthoester is of formula I, R is —(CH₂)_{b}—O—(CH₂)_{c}—; where band care both 2; R* is a C₂ alkyl; where the excipient is methoxy-polyethylene glycol (Mn 550), and the active agent comprises 2 wt % of the composition. In one embodiment, the active agent is granisetron.

In some embodiments of the methods of the present application, the polyorthoester used in the pharmaceutical composition of the present invention, as shown in formula I, formula II, formula III and formula IV, is one of alternating residues of a diketene acetal and a diol, with each adjacent pair of diketene acetal residues being separated by the residue of one polyol, preferably a diol.

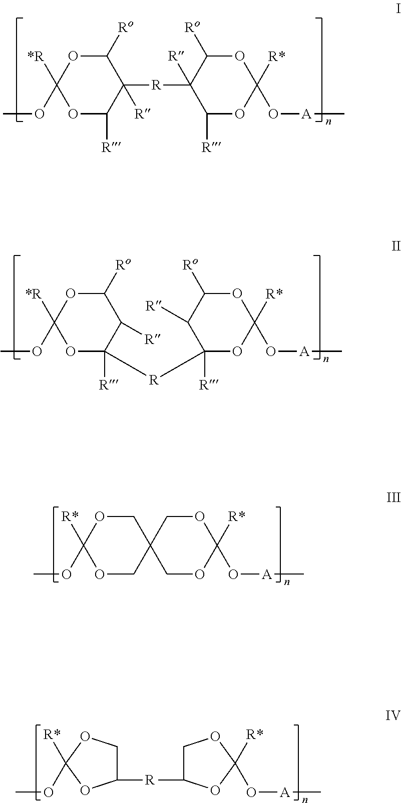

Polyorthoesters having a higher mole percentage of the “α-hydroxy acid containing” units will have a higher rate of bioerodibility. In one variation, the polyorthoesters are those in which the mole percentage of the “α-hydroxy acid containing” units is at least 0.01 mole percent, in the range of about 0.01 to about 50 mole percent, more preferably from about 0.05 to about 30 mole percent, for example from about 0.1 to about 25 mole percent, especially from about 1 to about 20 mole percent. The mole percentage of the “α-hydroxy acid containing” units appropriate to achieve the desired composition will vary from formulation to formulation.

In another variation, the polyorthoesters are those where: n is an integer of 5 to 1000; the polyorthoester has a molecular weight of 1000 to 20,000, preferably 1000 to 10,000, more preferably 1000 to 8000; R⁵ is hydrogen or methyl;

R⁶ is:

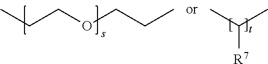

where s is an integer of 0 to 10, especially 1 to 4; t is an integer of 2 to 30, especially 2 to 10; and R⁷ is hydrogen or methyl;

R³ is:

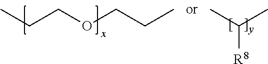

where x is an integer of 0 to 10, especially 1 to 4; y is an integer of 2 to 30, or 2 to 10; and R⁸ is hydrogen or methyl;

R⁴ is selected from the residue of an aliphatic diol of 2 to 20 carbon atoms, or 2 to 10 carbon atoms, interrupted by one or two amide, imide, urea or urethane groups;

the proportion of units in which A is R¹ is about 0.01-50 mol %, or 0.05-30 mol %, or 0.1-25 mol %;

the proportion of units in which A is R² is less than 20%, or less than 10%, especially less than 5%, and

the proportion of units in which A is R⁴ is less than 20%, less than 10%, or less than 5%.

### Excipients

In some embodiments of the above methods, the delivery vehicle of the pharmaceutical composition further comprises an excipient. The concentrations of the polyorthoester and the excipient in the delivery vehicle may vary. For example, the concentration of the excipient in the vehicle may be in the range of 1-99% by weight, preferably 5-80% weight, especially 20-60% by weight of the vehicle.

While the singular form is used to describe the polyorthoester and excipient in this application, it is understood that more than one polyorthoesters and excipients selected from the groups described above may be used in the delivery vehicle. It is also understood that while not required, other pharmaceutically acceptable inert agents such as coloring agents and preservatives may also be incorporated into the composition.

In some embodiments, when a polyorthoester is present in the pharmaceutical composition, the excipients are pharmaceutically acceptable and polyorthoester-compatible materials. In one embodiment, the excipients are liquid at room temperature, and are readily miscible with the polyorthoesters.

Suitable excipients include poly(ethylene glycol) ether derivatives having a molecular weight of between 200 and 4,000, such as poly(ethylene glycol) mono- or di-alkyl ethers, preferably poly(ethylene glycol)monomethyl ether 550 or poly(ethylene glycol)dimethyl ether 250; poly(ethylene glycol)copolymers having a molecular weight of between 400 and 4,000 such as poly(ethylene glycol-co-polypropylene glycol); propylene glycol mono- or di-esters of a C₂₋₁₉ aliphatic carboxylic acid or a mixture of such acids, such as propylene glycol dicaprylate or dicaprate; mono-, di- or tri-glycerides of a C₂₋₁₉ aliphatic carboxylic acid or a mixture of such acids, such as glyceryl caprylate, glyceryl caprate, glyceryl caprylate/caprate, glyceryl caprylate/caprate/laurate, glycofurol and similar ethoxylated tetrahydrofurfuryl alcohols and their C₁₋₄ alkyl ethers and C₂₋₁₉ aliphatic carboxylic acid esters; and biocompatible oils such as sunflower oil, sesame oil and other non- or partially-hydrogenated vegetable oils.

Certain formulations described herein are semi-solid pharmaceutical compositions that are easily syringable or injectable, meaning that they can readily be dispensed from a conventional tube of the kind well known for topical or ophthalmic formulations, from a needleless syringe, or from a syringe with a 16 gauge or smaller needle (such as 16-25 gauge), and injected subcutaneously, intradermally or intramuscularly. The formulations may be applied using various methods known in the art, including by syringe, injectable or tube dispenser.

### Active Agents

The pharmaceutical compositions described herein comprise one or more active agents to provide for a beneficial or useful result. Examples of active agents are described herein.

In some embodiments of the methods or compositions of the present application, the active agent is a local anesthetic. Non-limiting examples of local anesthetics include amide- or anilide-type local anesthetics such as bupivacaine, dibucaine, mepivacaine, procaine, lidocaine, tetracaine and the like. In some embodiments, the active agent is mepivacaine or buprenorphine.

In some embodiments of the methods or compositions of the present application, the active agent is an anti-CINV (chemotherapy-induced nausea and vomiting) agent, such as a selective 5-hydroxytryptamine 3 (5-HT₃) receptor antagonist (for example, granisetron (Kytril®), ondansetron (Zofran®), dolasetron (Anzemet®) and palonosetron (Aloxi®, MGI Pharma)). Compositions having a (5-HT₃) receptor antagonist are useful for the prevention, reduction or alleviation of acute and delayed chemotherapy-induced nausea and vomiting (CINV) following a course of emetogenic chemotherapy. In some embodiments, the composition is administered by subcutaneous injection.

In some embodiments, the APF530 pharmaceutical composition comprises a 5-HT₃ receptor antagonist, a polymer susceptible to hydrolysis and a pharmaceutically acceptable liquid excipient; wherein the composition, when administered in a single dosage, provides a release profile of the 5-HT₃ receptor antagonist that tracks the profile of an incidence of vomiting; and wherein the composition provides a level of the 5-HT₃ receptor antagonist over 24 hours to provide a % Cₘₐₓ profile that is within 20% of the profile in the incidence of vomiting, provides a sustained levels over 96 hours to provide a % Cₘₐₓ profile that is within 10% of the profile in the incidence of vomiting, and provides essentially no 5-HT₃ receptor antagonist concentration in plasma at about 144 hours. In some embodiments, the level of the 5-HT₃ receptor antagonist over 24 hours, is substantial and may be measured experimentally as provided herein, to provide a % Cₘₐₓ profile that is within 20% of the profile in the incidence of vomiting, provides a sustained levels over 96 hours to provide a % Cₘₐₓ profile that is within 10% of the profile in the incidence of vomiting, and provides essentially no detectable 5-HT₃ receptor antagonist concentration in plasma at about 144 hours.

In some embodiments, the 5-HT₃ receptor antagonist is granisetron. In some embodiments, the effective amount of the 5-HT₃ receptor antagonist is a single dose of about 5 mg to about 10 mg. In some embodiments, the administration by subcutaneous injection is performed at about three hours, two hours, one hour or 30 minutes before chemotherapy. In a variation of the above, the administration by subcutaneous injection is performed at about 30 minutes before chemotherapy. In another variation, the subcutaneous injection is performed over about 30 seconds. In yet another variation, the 5-HT₃ receptor antagonist is granisetron and the effective amount of granisetron is about 5 mg.

In other embodiments of the methods of the present application, the APF530 pharmaceutical composition provides a substantial level of the 5-HT₃ receptor antagonist over 24 hours to provide a % Cₘₐₓ profile that is within 10% of the profile in the incidence of vomiting. In some embodiments, the composition provides sustained levels over 96 hours to provide a % Cₘₐₓ profile that is within 5% of the profile in the incidence of vomiting. In some embodiments, the composition a substantial level of the 5-HT₃ receptor antagonist over 24 hours to provide a % Cₘₐₓ profile that is within 10% of the profile in the incidence of vomiting, provides a sustained levels over 96 hours to provide a % Cₘₐₓ profile that is within 5% of the profile in the incidence of vomiting, and provides essentially no 5-HT₃ receptor antagonist concentration in plasma at about 144 hours. In some embodiments, the administration of an effective amount of the 5-HT₃ receptor antagonist to a patient result in further reducing the incidence of reported headaches to less than about 40%, 30%, 20% or about 10% in patients receiving chemotherapy. In some embodiments, the incidence of reported headaches is less than about 20% in patients receiving chemotherapy.

In another embodiment, there is provided the above noted method, wherein the APF530 pharmaceutical composition, when administered to a patient, releases a substantial level of the 5-HT₃ receptor antagonist over 24 hours to provide a % Cₘₐₓ profile that is within 10% of the profile in the incidence of vomiting, provides a sustained levels over 96 hours to provide a % Cₘₐₓ profile that is within 5% of the profile in the incidence of vomiting, and provides essentially no 5-HT₃ receptor antagonist concentration in plasma at about 144 hours. In one variation, the administration of an effective amount of the 5-HT₃ receptor antagonist to a patient results in further reducing the incidence of reported headaches to less than about 40%, 30%, 20% or about 10% in patients receiving chemotherapy. In another variation, the incidence of reported headaches is less than about 20% in patients receiving chemotherapy. In yet another variation, the administration of an effective amount of the 5-HT₃ receptor antagonist to a patient result in a statistically significant reduction in the incidence of vomiting following emetogenic chemotherapy than that of the administration of palonosetron by iv infusion.

In a variation of the above, the 5-HT₃ receptor antagonist is granisetron. In a particular variation, the fraction of granisetron is from 0.1% to 80% by weight of the composition. In yet another variation, the fraction of granisetron is about 1% to 5% by weight of the composition.

In some embodiments of the methods of the present application, the pharmaceutical composition comprises 2% granisetron, and a semi-solid delivery vehicle comprising,

(i) 78.4 weight % of the polyorthoester of formula I:

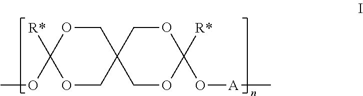

where:

R* is a C₂ alkyl;

n is an integer of at least 5; and

A is R¹ or R³ where R¹ is:

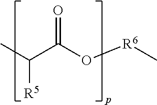

where: p is 2; R⁵ is hydrogen; and

R⁶ is:

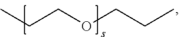

where: s is 3; and R³ is:

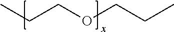

where x is 3;

where the polyorthoester comprises 47.4 mole % DETOSU, 42.1 mole % TEG, and 10.5 mole % of the A units are of the formula R¹; and

(ii) a pharmaceutically acceptable, polyorthoester-compatible liquid excipient that is 19.6 weight % MPEG 550 (methoxy-polyethylene glycol, Mn 550). Delivery of Controlled-Release Granisetron:

The present application further relates to a method for the treatment or prevention of emesis in a patient which comprises administering a pharmaceutical composition comprising a 5-HT₃ antagonist, wherein the 5-HT₃ antagonist minimize the side effects of nausea and/or emesis associated with other pharmacological agents, wherein the stability of the composition enhanced by a method of this invention. In a particularly preferred aspect, the 5-HT₃ antagonist is granisetron.

As used herein, the term “emesis” includes nausea and vomiting. The 5-HT₃ antagonists in the semi-solid injectable form of the present invention are beneficial in the therapy of acute, delayed or anticipatory emesis, including emesis induced by chemotherapy, radiation, toxins, viral or bacterial infections, pregnancy, vestibular disorders (e.g. motion sickness, vertigo, dizziness and Meniere's disease), surgery, migraine, and variations in intracranial pressure. In one aspect of each of the above, the 5-HT₃ antagonist is granisetron.

The semi-solid injectable form of granisetron of the present invention is prepared by incorporating the antiemetic agent into the delivery vehicle in a manner as described above. The concentration of granisetron may vary from about 0.1-80 wt. %, preferably from about 0.2-60 wt. %, more preferably from about 0.5-40 wt. %, most preferably from about 1-5 wt. %, for example, about 2-3 wt. %. The semi-solid composition is then filled into a syringe with a 16-25 gauge needle, and injected into sites that have been determined to be most effective. The semi-solid injectable composition of the present invention can be used for controlled delivery of both slightly soluble and soluble antiemetic agents.

In one particular aspect, granisetron may be used in the form of a salt or salts or mixtures of granisetron and the salt of granisetron. Suitable pharmaceutically acceptable salts of granisetron of use in the present invention include acid addition salts which may, for example, be formed by mixing a solution of granisetron with a solution of a pharmaceutically acceptable non-toxic acid such as hydrochloric acid, iodic acid, fumaric acid, maleic acid, succinic acid, acetic acid, citric acid, tartaric acid, carbonic acid, phosphoric acid, sulfuric acid and the like.

### Experimental

### Preparation of Formulation

The various polymers comprising an active agent may be prepared using the procedures as taught herein. In one example, the active agent is granisetron. In one example, the polymer (Polymer A) comprises 47.4 mole % DETOSU, 42.1 mole % TEG, and 10.5 mole % of the latent acid. The Mw is 5,900-8,100 daltons and the Mn is 2,900-4,000 daltons. In another series of polymer compositions that are prepared, the polymer may comprise of about 40-60 mole % DETOSU, 40-60 mole % TEG, and 5-20 mole % latent acid.

In one example, Formulation F comprises 78.4 weight % Polymer A, 19.6 weigh % MPEG 550 (methoxy-polyethylene glycol, Mn 550) and 2% granisetron.

Formulation F comprises of 2% (w/w) granisetron in a proprietary triethylene glycol-polyorthoester (“TEG-POE”) polymer with methoxypolyethylene glycol (“MPEG”) as an excipient to reduce viscosity. The product is supplied as a clear, sterile viscous liquid in pre-filled syringes. Formulation F is manufactured under GMP conditions by dissolving crystalline granisetron in a mixture of MPEG and TEG-POE polymer. Currently the bulk product is sterilized by gamma irradiation and is aseptically filled into syringes.

Formulation F with enhanced stability (Formulation FR) is manufactured by dissolving crystalline granisetron in a mixture of MPEG and TEG-POE polymer, followed by a heat treatment. A typical heat treatment consists of heating the formulation to temperatures such as 90-95° C. over 24 h under a dynamic inert gas environment. The controlled thermal exposure of the composition or formulation is accomplished in a cylindrical reactor which is jacketed to allow for temperature control over the vessel contents. The formulation is continuously stirred with an overhead agitator under an inert atmosphere of nitrogen or argon. Higher temperatures for short time periods or lower temperatures for longer time periods will allow the formulation to attain a reduced molecular weight and viscosity which will remain stable at room temperature.

An alternative process may consist of carrying out similar heat treatments on the polyorthoester (such as TEG-POE) or polyorthoester and excipient (such as MPEG) prior to the addition of the active ingredient (such as granisetron) such that a composition of enhanced stability as disclosed herein, is obtained.

Another alternative composition as described above, comprises a suspension of the active ingredient, such as granisetron.

The methods of the present application may be particularly useful in enhancing the stability of the pharmaceutical compositions comprising Polymer A, granisetron free base and MPEG 550 according to the following table:

| | | | MPEG 550 as a | |
|---|---|---|---|---|
| | | | percent of | |
| | | | Polymer A + | Granisetron |
| | Polymer A | MPEG 550 | MPEG 550 | free base |
| Formulation | (weight %) | (weight %) | (weight %) | (weight %) |
| Formulation F1 | 93.1% | 4.9% | 5% | 2.0% |
| Formulation F2 | 88.2% | 9.8% | 10% | 2.0% |
| Formulation F | 78.4% | 19.6% | 20% | 2.0% |
| (APF530) | | | | |
| Formulation FR | 78.4% | 19.6% | 20% | 2.0% |
| (heat-treated, | | | | |
| enhanced stability) | | | | |
| Formulation F3 | 68.6% | 29.4% | 30% | 2.0% |
| Formulation F4 | 58.8% | 39.2% | 40% | 2.0% |
| Formulation F5 | 49.0% | 49.0% | 50% | 2.0% |
| Formulation F6 | 39.2% | 58.8% | 60% | 2.0% |
| Formulation F7 | 29.4% | 68.6% | 70% | 2.0% |
| Formulation F8 | 19.6% | 78.4% | 80% | 2.0% |
| Formulation F9 | 9.8% | 88.2% | 90% | 2.0% |
| Formulation F10 | 0% | 98.0% | 100% | 2.0% |

APF112A (Polymer A containing mepivacaine) and APF530 are viscous, liquid polyorthoester formulations based on APF135 (Polymer A) and MPEG-550, containing the active pharmaceutical ingredients, 3% mepivacaine and 2% granisetron, respectively. Results from stability studies of these formulations have shown that the molecular weight of the polyorthoesters remains unchanged for at least 2 years, when sealed syringes containing these materials are stored at 2-8° C.

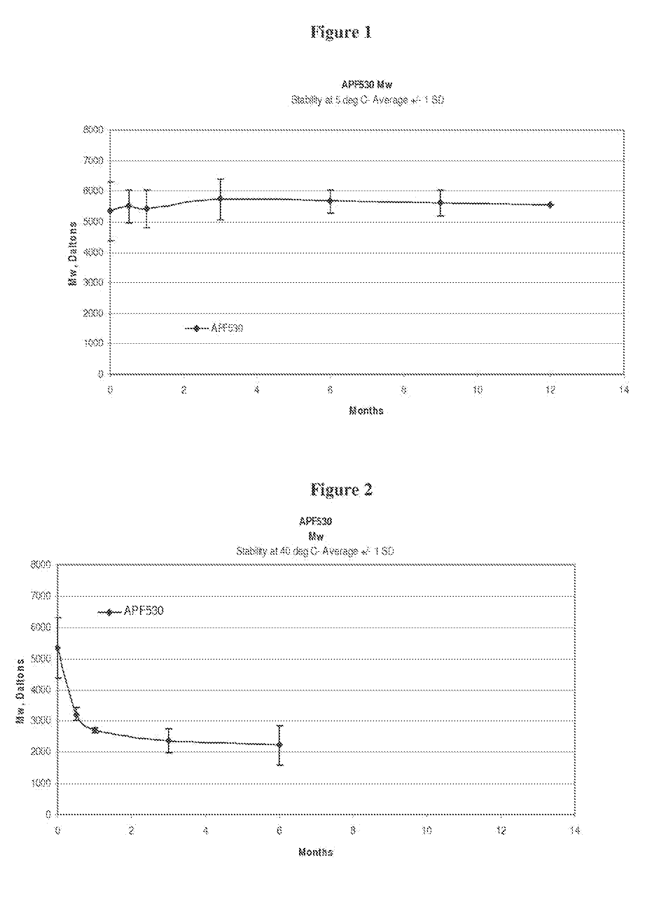

Gel Permeation Chromatography (GPC) is a form of liquid chromatography in which the solute molecules are separated as a result of their permeation into a solvent-filled matrix in the column packing. The larger molecules pass quickly through the column, while smaller molecules diffuse in and out of the pores and emerge later. Since the size of the molecule is related to its molecular weight, the elution time of a given peak provides a measurement of a molecule's molecular weight. The retention time or retention volume is acquired for a series of molecular weight standards as well as samples. General GPC utilizes polystyrene standards with molecular weights ranging from 1,000,000 to 500 Daltons. Intermediate GPC utilizes poly(ethylene)glycol standards with molecular weights ranging from 15,000 to 100 Daltons. Samples elute from a two column set utilizing THF mobile phase and a differential refractive index detector. The calibration curve is obtained by plotting the logarithmic molecular mass versus retention time or retention volume. Based on the calibration curve of the standards, the molecular weight of the formulation is determined. The formulation viscosity and in vitro release of the active ingredient are also stable under these storage conditions. Viscosity is the measure of the internal friction of a fluid, caused by molecular attraction, which makes it resist a tendency to flow. The viscosity is measured by a Brookfield viscometer which also consists of a programmable water bath set at 37° C. Using a specified spindle, the initial spindle speed (rpm) is determined by briefly rotating the sample at different speeds to obtain the desired torque of approximately 10% of the maximum torque. The shear stress is then measured through the torque range of 10-100% to yield at least 10 measurements. The viscosity is calculated from the shear stress and the shear rate for each individual measurement, and the data is plotted as viscosity versus spindle speed (rpm). From the best fit for this plot, the viscosity at 10 rpm is calculated and reported. In Vitro Release (IVR) measures the amount of drug released from the formulation during specified time intervals. Phosphate buffer is added to a specified amount of formulation which is then incubated at 37° C. At specified time intervals, the receptor fluid is removed and then replenished. The removal and replenishment of receptor fluid continues until the formulation is no longer visible. The removed receptor fluid is analyzed by High Performance Liquid Chromatography (HPLC) to determine the drug content. A profile is then generated as a function of time in which the percent release is calculated over the incubation time. However, when the syringes are stored at 40° C., the molecular weight of the formulations reduces over a two-four week period to a lower level, after which no further reduction is seen. A corresponding initial reduction in the formulation viscosity and increase in the in vitro release of the active ingredient, followed by plateaus at new levels, are also observed. FIGS. 1 and 2 illustrate the molecular weight stability data for APF530.

Without being bound by a theory or by a specific mechanism provided herein, a possible hypothesis for this behavior is the reactive nature of polyorthoesters to water in these compositions. Water, at low levels (0.1% or less), has been shown to be present in the formulations, possibly introduced due to incidental exposure during the manufacturing process and/or from the inherently low levels present in the viscosity modifier, MPEG 550. Under certain conditions this water may react with the polyorthoester and contribute to the reduction in its molecular weight. Such a reaction would be expected to be sufficiently slow at 2-8° C. due to reduced diffusion and kinetics at low temperatures, which provide a possible explanation of the stability of the formulation (FIG. 1) under these conditions. On the other hand, at 40° C., diffusion and kinetics of the reaction would be favored, which might cause the reduction in molecular weight that takes place in a 2-4 week period for the formulation (FIG. 2). It is hypothesized that once most of the water is consumed, with minimum or no external water entering the sealed syringes, no further reaction would be expected to take place and hence, further reduction in the molecular weight is largely absent. Formulations in syringes stored at 25° C. display this reduction in molecular weight over a longer period of time; however, they eventually arrive at the same lower molecular weight plateau seen for the materials stored at 40° C. These formulations are transported and stored at 2-8° C. until ready for use.

This feature of the present polyorthoesters can be exploited by preparing various formulations that are blended and then heated at appropriate times and temperatures. Without being bound by any theory provided herein, it is believed that the heat treatment method result in the reaction or consumption of the water that is present, resulting in the formation of a new and stable formulation. Higher temperatures such as 90-95° C. over 24 h, and the use of processing aids such as a dynamic (flowing) inert gas environment or the application of vacuum, are contemplated to cause the formulations to quickly reach the stable molecular weight plateaus, similar to the ones seen in the stability studies. The compositions of the present application that are heated according to the methods described herein may also be referred to as being heat treated.

The controlled thermal exposure of the composition or formulation is accomplished in a cylindrical reactor which is jacketed to allow for temperature control over the vessel contents. The formulation is continuously stirred with an overhead agitator under an inert atmosphere of nitrogen or argon. Higher temperatures for short time periods or lower temperatures for longer time periods will allow the formulation to attain a reduced molecular weight and viscosity which will remain stable at room temperature.

With most of the water consumed as a result of this process, such formulations would no longer need to be stored at 2-8° C. and may be kept at room temperature instead, making them easier to store and transport. Moreover, the reduction in molecular weight will cause a corresponding reduction in viscosity, which will improve the ease of administering the formulations through smaller needles. There would also be an expected increase in the in vitro release of the active ingredient; however, the molecular weight and viscosity are still sufficiently maintained such that control of the release would also be maintained. Depending on the active ingredient used, a shorter duration of release may indeed be desired and beneficial. In addition, the plateau of molecular weight, viscosity, and in vitro release may be adjusted by a suitable choice of the starting molecular weight of the polymer.

In one aspect, the stabilized pharmaceutical composition prepared according to the method provides a composition that remains stable for storage at room temperature for at least 1 month, at least 3 months, at least 5 months, at least 8 months and at least 12 months. The stabilized pharmaceutical composition remains stable over the above period of time without a 15%, 10%, 5%, 3% or 2% reduction in the molecular weight of the polymer as measured by GPC. The stabilized pharmaceutical composition as prepared herein remains stable over the above period of time without a 15%, 10%, 5%, 3% or 2% change in viscosity as measured by dynamic viscosity, such as by using a Brookfield viscometer. In certain embodiments, the stabilized pharmaceutical compositions as prepared herein provide a 30%, a 20%, a 10% or a 5% shorter time for the release of the active agent than the corresponding pharmaceutical composition that is not heat treated.

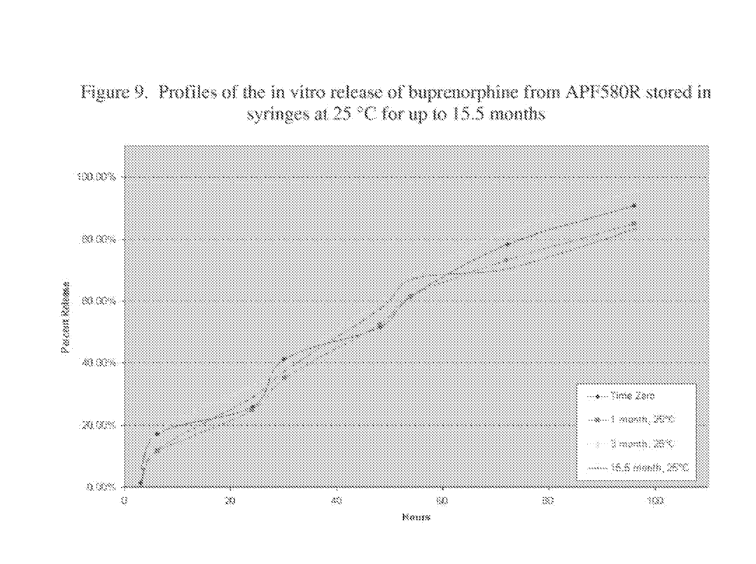

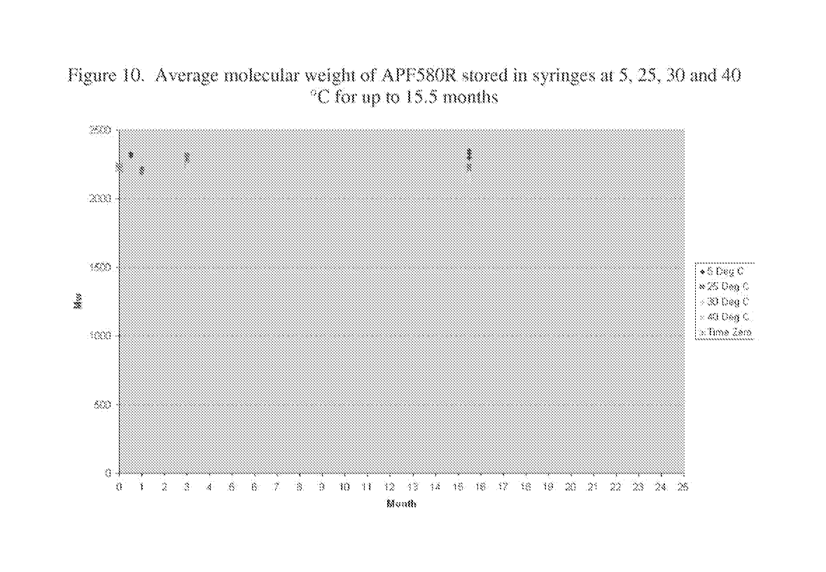

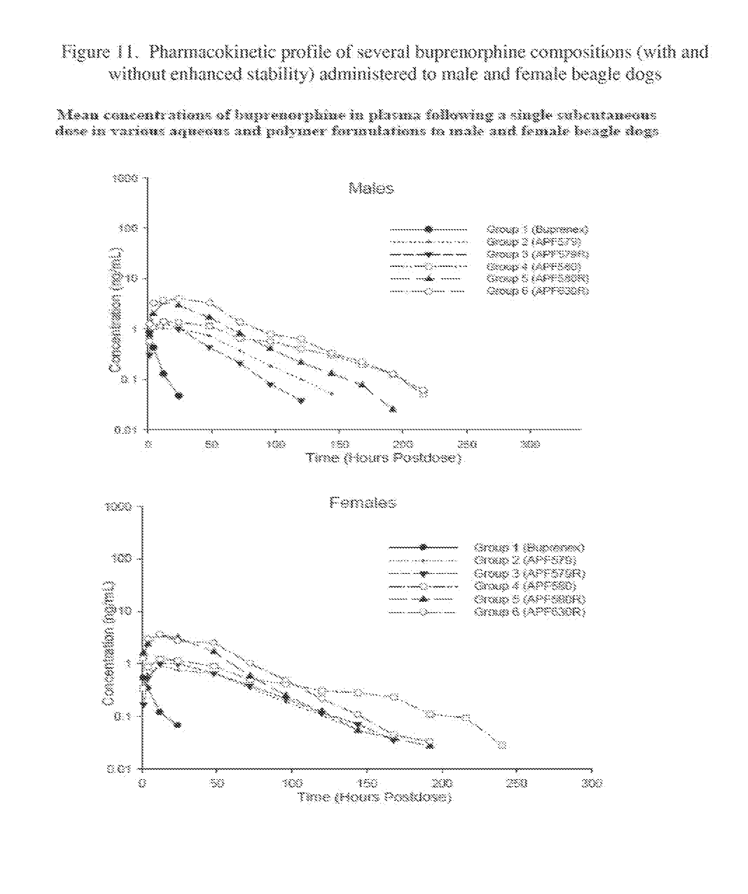

We have prepared similar formulations containing buprenorphine, 0.25%, 0.50%, 0.75%, 1.00% and 2.00%, that correspond to APF636R, APF626R, APF637R, APF579R and APF580R, respectively, which have been heated for 24 h at 90° C. under argon. The resulting heat treated compositions are lower in molecular weight and viscosity as compared to the same compositions that are not heat treated, and the heat treated compositions were shown to be unchanged or stable when stored in syringes at 25° C. based on 15-month stability data (FIGS. 9 and 10). These formulations also show a more desirable, shorter duration of release of the active ingredient than the corresponding formulations that were prepared without the heat treatment process.

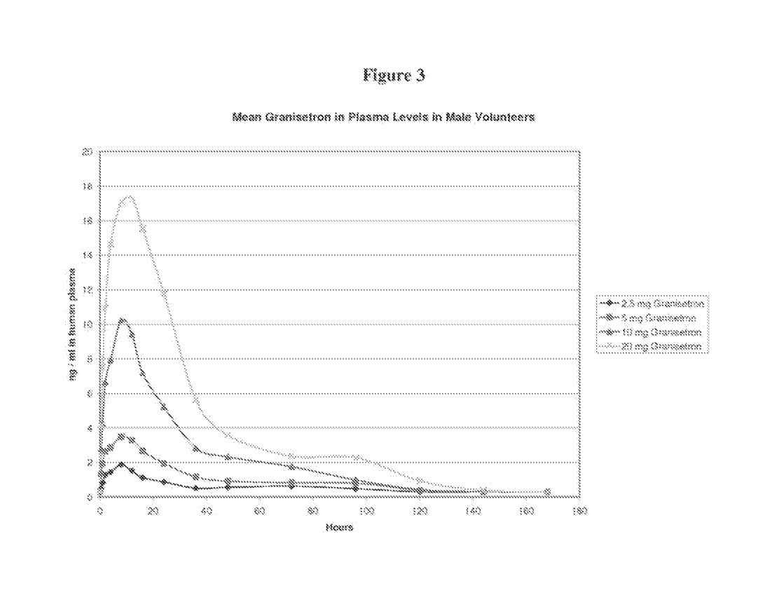

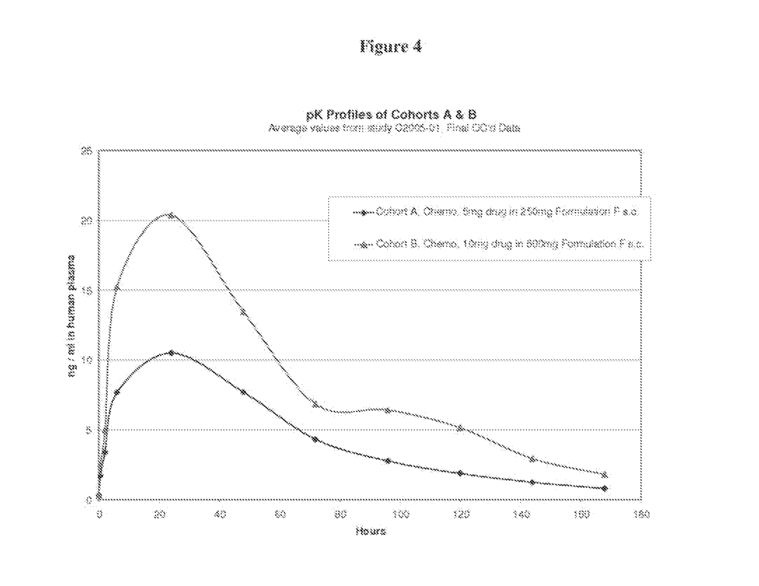

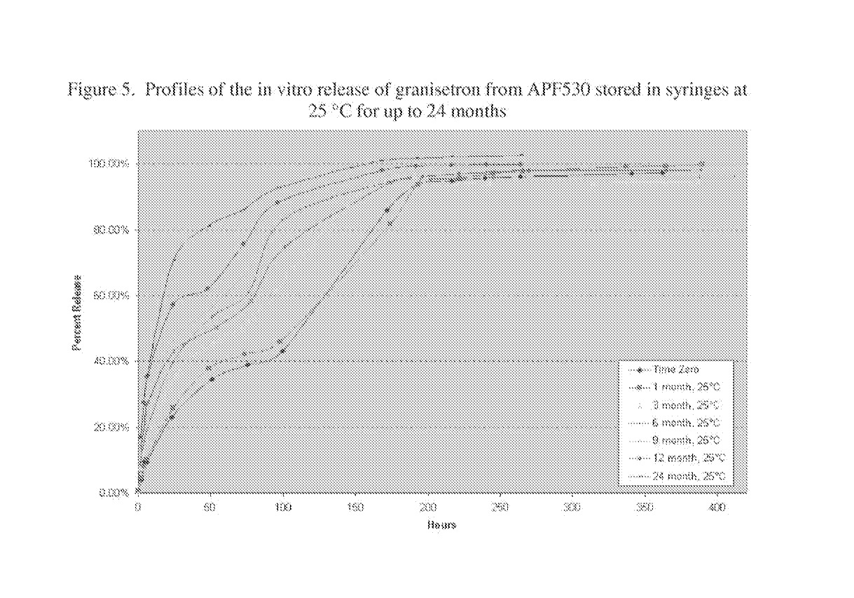

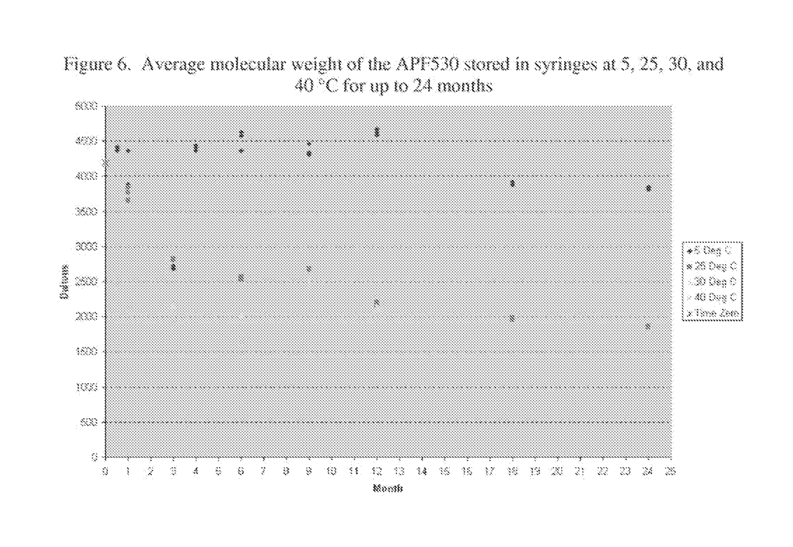

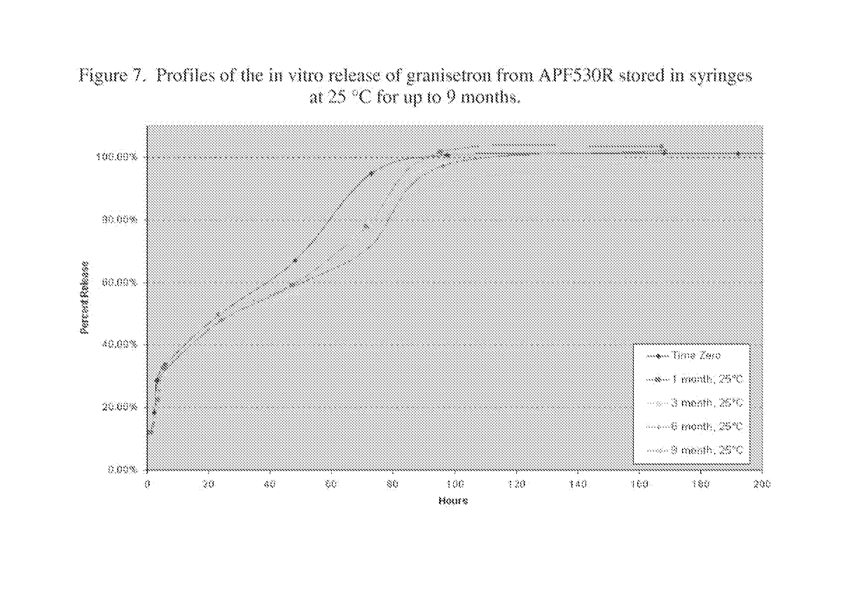

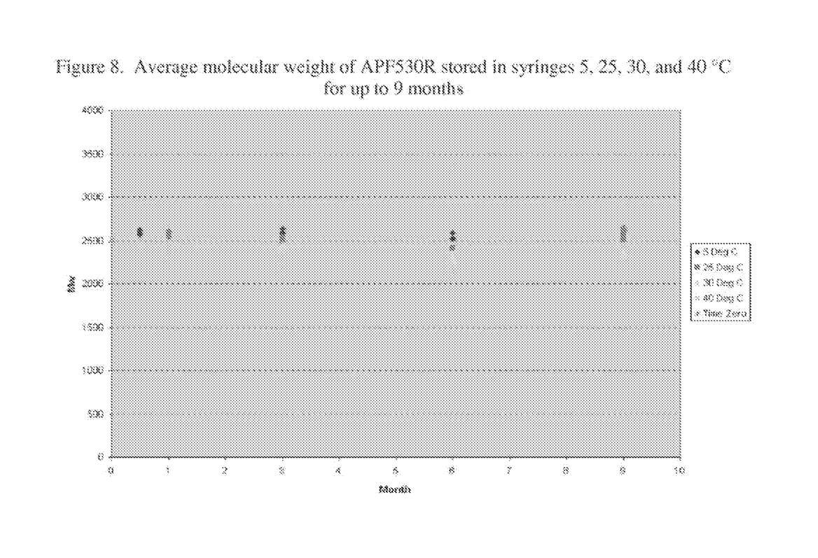

Phase 3 data on APF530, stored at 2-8° C. have shown the presence of the active ingredient, granisetron, for five days in human plasma, and a corresponding control of emesis for that duration for patients undergoing chemotherapy. The novel and stable granisetron-containing formulations prepared using the process described above can be stored at room temperature (25° C., as seen from the 9-month stability data depicted for APF530R in FIGS. 7 and 8), are easier to administer through smaller gauge needles due to reduced viscosity, and may provide release from nausea and vomiting for at least 5 days. While the clearance of granisetron is variable between individuals, it is clear from the data from human trials that there is impaired clearance in chemo-patients probably a consequence of disease, age, and concomitant chemotherapy (see FIGS. 3 and 4). This impaired clearance would be expected to mitigate the effect of any increase in the drug release rate and thus allow for therapeutic benefit over 5 days.

The foregoing is offered primarily for purposes of illustration. It will be readily apparent to those skilled in the art that the molecular structures, proportions of the various components in the delivery vehicle or pharmaceutical composition, method of manufacture and other parameters of the invention described herein may be further modified or substituted in various ways without departing from the spirit and scope of the invention. For example, the temperature, pressure and time may vary according to and depending upon factors, such as the particular polymers and active compounds in the formulation, and such expected variations or differences are contemplated in accordance with the objects and practices of the present invention. It is intended, therefore, that the invention be defined by the scope of the claims which follow and that such claims be interpreted as broadly as is reasonable.

What is claimed is:

1. A method of enhancing the stability of a sustained release pharmaceutical composition comprising an active agent and a polymer, wherein the method comprises heating the pharmaceutical composition at an elevated temperature for a sufficient period of time to provide a more stable pharmaceutical composition than that of the unheated pharmaceutical composition, when stored at room temperature.

2. The method of claim 1, wherein the heating of the pharmaceutical composition is performed under an inert gas.

3. The method of claim 1, wherein the heating of the pharmaceutical composition results in the reduction of water content from the composition.

4. The method of claim 1, wherein the heating of the pharmaceutical composition is performed above 50° C. for at least one hour.

5. The method of claim 1, wherein the heating of the pharmaceutical composition is performed above 50° C. for at least 3 hours.

6. The method of claim 1, wherein the heating of the pharmaceutical composition is performed at about 90° C. for at least 3 hours.

7. The method of claim 1, wherein the heating of the pharmaceutical composition is performed at 90° C. for 24 hours.

8. The method of claim 1, wherein the polymer is a bioerodible polymer, or a polymer that is susceptible to hydrolysis.

9. The method of claim 1, wherein the polymer is a polyorthoester.

10. A method of enhancing the stability of a sustained release pharmaceutical composition comprising a polymer, wherein the method comprises heating the pharmaceutical composition at an elevated temperature for a sufficient period of time to provide a more stable pharmaceutical composition than that of the unheated pharmaceutical composition, when stored at room temperature.

11. The method of claim 10, wherein the polymer is a polyorthoester polymer, and the heating of the pharmaceutical composition is performed above 50° C. for at least 3 hours.

12. A method of enhancing the stability of a sustained release pharmaceutical composition comprising an active agent and a polyorthoester polymer, wherein the method comprises treating the pharmaceutical composition under one or more of the following conditions: an elevated temperature, a sufficient period of time, an inert gas, and a reduced pressure.

13. A method of preparing a sustained release pharmaceutical composition with enhanced stability wherein the method comprises treating the pharmaceutical composition under one or more of the following conditions: an elevated temperature, a sufficient period of time, an inert gas, and a reduced pressure, and wherein the pharmaceutical composition comprises an active agent and a polyorthoester polymer.

14. The method of claim 1, wherein the temperature is at least about 80° C.

15. The method of claim 14, wherein the elevated temperature is maintained for a period of time of at least 24 hours.

16. The method of claim 15, wherein after the treatment, the average molecular weight of the polymer is reduced.

17. The method of claim 1, wherein the polymer is selected from the group consisting of: 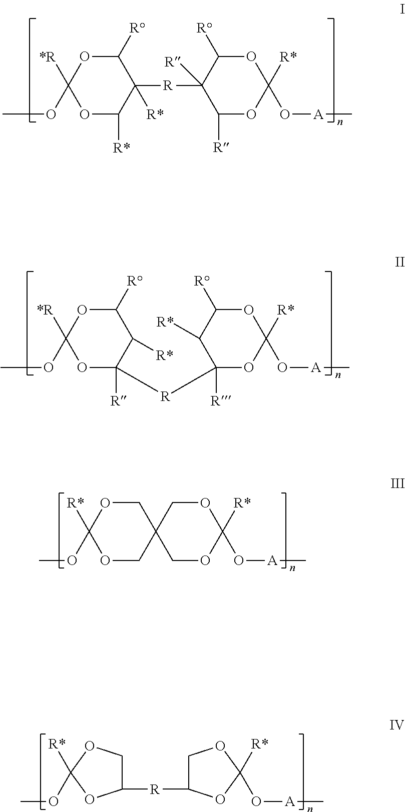
where:
R is a bond, —(CH₂)a-, or —(CH₂)b-O—(CH2)c-; where a is an integer of 1 to 10, and b and c are independently integers of 1 to 5;
R* is a C₁₋₄ alkyl;
R^{o}, R″ and R′″ are each independently H or C₁₋₄ alkyl;
n is an integer of at least 5; and
A is R¹, R², R³, or R, where
R¹ is: 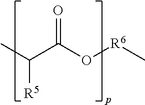
where:
p is an integer of 1 to 20;
R⁵ is hydrogen or C₁₋₄ alkyl; and
R⁶ is: 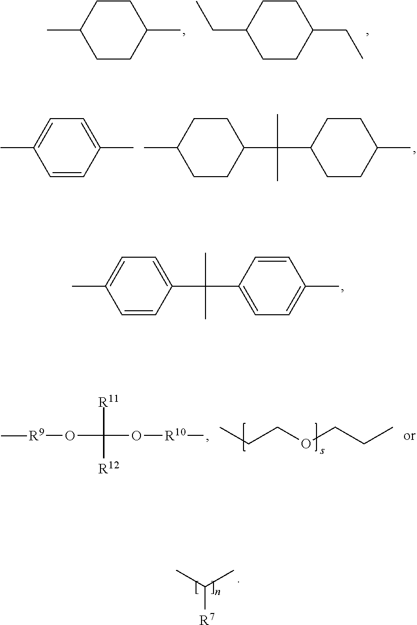
where:
s is an integer of 0 to 30;
t is an integer of 2 to 200; and
R⁷ is hydrogen or C₁₋₄ alkyl;
R² is: 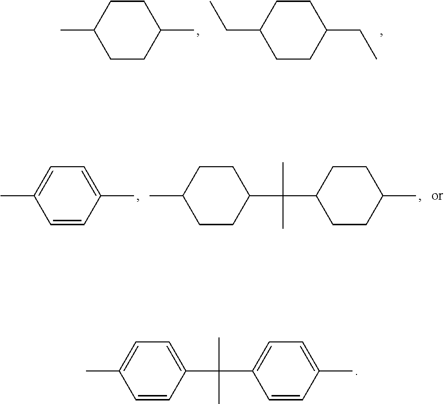
R³ is: 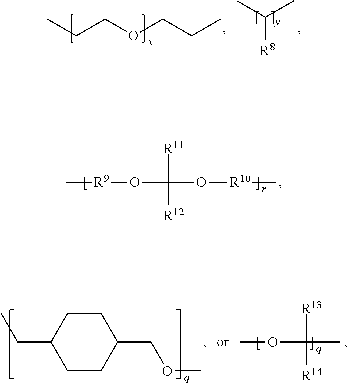
where:
x is an integer of 0 to 100;
y is an integer of 2 to 200;
q is an integer of 2 to 20;
r is an integer of 1 to 20;
R⁸ is hydrogen or C₁₋₄ alkyl;
R⁹ and R¹⁰ are independently C₁₋₁₂ alkylene;
R¹¹ is hydrogen or C₁₋₆ alkyl and R¹² is C₁₋₆ alkyl; or R¹¹ and R¹² together are C₃₋₁₀ alkylene; and
R⁴ is the residue of a diol containing at least one functional group independently selected from amide, imide, urea, and urethane groups;
in which at least 0.01 mol percent of the A units are of the formula R¹.

18. The method of claim 1, wherein the composition comprises granisetron, a semi-solid delivery vehicle and a pharmaceutically acceptable liquid excipient; wherein:
(A) the semi-solid delivery vehicle, comprises:
(i) a polyorthoester of formula I 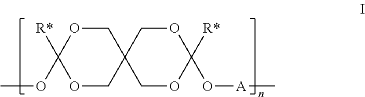
where:
R* is a C₁₋₄ alkyl;
n is an integer of at least 5; and
A is R¹, R², R³, or R⁴, where 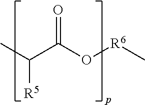
where:
p is an integer of 1 to 20;
R⁵ is hydrogen or C₁₋₄ alkyl; and
R⁶ is: 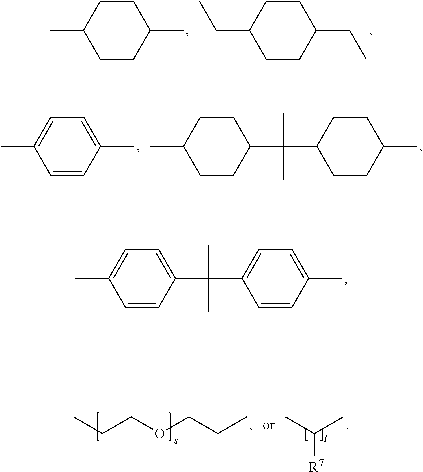
where:
or s is an integer of 0 to 30;
t is an integer of 2 to 200; and
R⁷ is hydrogen or C₁₋₄ alkyl;
R² is: 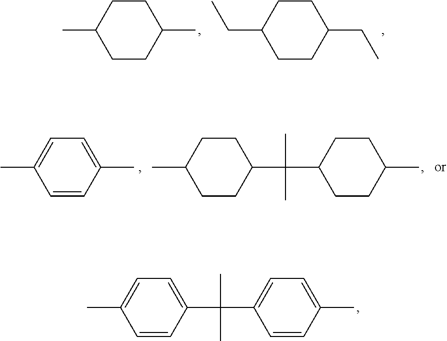
R³ is: 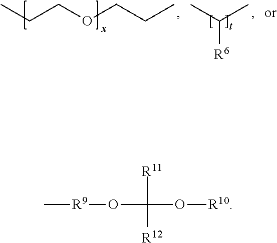
where:
x is an integer of 0 to 30;
y is an integer of 2 to 200;
R⁸ is hydrogen or C₁₋₄ alkyl;
R⁹ and R¹⁰ are independently C₁₋₁₂ alkylene;
R¹¹ is hydrogen or C₁₋₆ alkyl and R¹² is alkyl; or R¹¹ and R¹² together are C₃₋₁₀ alkylene; and
R⁴ is the residue of a diol containing at least one functional group independently selected from amide, imide, urea, and urethane groups;
in which at least 0.01 mol percent of the A units are of the formula R¹; and
(ii) a pharmaceutically acceptable, polyorthoester-compatible liquid excipient selected from polyethylene glycol ether derivatives having a molecular weight between 200 and 4000, polyethylene glycol copolymers having a molecular weight between 400 and 4000, mono-, di-, or tri-glycerides of a C₂₋₁₉ aliphatic carboxylic acid or a mixture of such acids, alkoxylated tetrahydrofurfuryl alcohols and their C₁₋₄ alkyl ethers and C₂₋₁₉ aliphatic carboxylic acid esters, and biocompatible oils.

19. The method of claim 17, wherein the pharmaceutical composition comprises:
(i) 2% granisetron;
(ii) 78.4 weight % of the polyorthoester of formula 1: 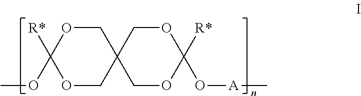
where:
R* is a C₂ alkyl;
n is an integer of at least 5; and
A is R¹ or R³ where R¹ is: 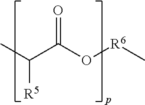
where:
p is 2;
R⁵ is hydrogen; and
R⁶ is: 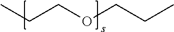
where:
s is 3; and
R³ is: 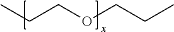
where x is 3;
where the polyorthoester comprises 47.4 mole % DETOSU, 42.1 mole % TEG, and 10.5 mole % of the A units are of the formula R¹ and
(iii) a pharmaceutically acceptable, polyorthoester-compatible liquid excipient that is 19.6 weight % MPEG 550 (methoxy-polyethylene glycol, Mn 550).

20. A stabilized pharmaceutical composition prepared by the method of claim 1.
